(19) United States
(12) Patent Application Publication
(10) Pub. No.: US 2008/0027140 A1
(43) Pub. Date: Jan. 31, 2008

(54) Combinations of Serotonin Reuptake Inhibitors and Imidazoline I ² Agonists

(75) Inventors: Gerd Bartoszyk, Weiterstadt (DE); Christoph Van Amsterdam, Darmstadt (DE); Henning Boettcher, Darmstadt (DE); Christoph Seyfried, Seeheim-Juenheim (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: MERCK PATENT GMBH, Darmstadt (DE)

(21) Appl. No.: 10/586,457

(22) PCT Filed: Dec. 15, 2004
(86) PCT No.: PCT/EP04/14284

§ 371 Date: Jul. 18, 2006

(30) Foreign Application Priority Data
Jan. 19, 2004 (DE) 10 2004 002 638.6

(51) Int. Cl. A61K 31/135; A61P 25/00

(52) U.S. Cl. 514/646

(57) ABSTRACT

The present invention relates to combinations of serotonin reuptake inhibitors and imidazoline I₂ agonists as separate chemical units or both properties combined in a single molecule and to the use thereof for the preparation of medicaments for the treatment of depression, obsessive-compulsive disorders (OCDs), obsessive-compulsive spectrum disorders (OCSDs) and other anxiety states.

The present invention relates to combinations of serotonin reuptake inhibitors (SRIs) and imidazoline I₂ agonists as separate chemical units or both properties combined in a single molecule and to the use thereof for the preparation of medicaments for the treatment of depression, obsessive-compulsive disorders (OCDs), obsessive-compulsive spectrum disorders (OCSDs) and other anxiety states.

Serotonin reuptake inhibitors, such as, for example, fluoxetine, sertraline, paroxetine, fluvoxamine or citalopram, are standard therapeutic agents for the treatment of depression, such as unipolar depression, bipolar depression, dysthymia or secondary depression in connection with physical diseases, obsessive-compulsive disorders, obsessive-compulsive spectrum disorders (for example Pigott T A, Seay S M (1999). J Clin Psychiatry 60: 101-106) and other anxiety states, such as generalised anxiety, post-traumatic stress disorder, social anxiety or panic disorder. They are furthermore employed for the treatment of eating disorders, such as anorexia nervosa and bulimia nervosa, premenstrual syndrome and premenstrual dysphoria (for example Masand and Gupta, Harv Rev Psychiatry 1999, 7: 69-84; Pigott and Seay, J Clin Psychiatry 1999, 60: 101-106; Bailer et al., Wiener Klein Wochenschr 2000, 112: 865-785; Zohar and Westenberg, Acta Psychiatr Scand 2000, 403 (Suppl.): 39-49; Hollander et al., J Clin Psychiatry 2002, 63 (Suppl 6): 20-29; Pearlstein and Yonkers, Expert Opin Pharmacother 2002, 3: 979-991; Vaswani et al., Prog Neuropsychopharmacol Biol Psychiatry 2003, 27, 85-102).

The most-used serotonin reuptake inhibitor is fluoxetine. The preparation is described, for example, in DE-A 25 00 110 or in U.S. Pat. No. 4,314,081 (Malloy and Schmiegel, 1975, 1982).

Imidazoline I₂ agonists, such as, for example, 2-BFI, LSL 60101, LSL 61122, BU224, BU236, RX801077, RX 821029, tracizoline or benazoline, have been proposed for the treatment of depression and eating disorders (for example Piletz et al., Crit Rev Neurobiol 1994, 9: 29-66; Alemany et al., Eur J Pharmacol 1995, 280: 205-210; Brown et al., Br J Pharmacol 1995, 116: 1737-1744; Carpene et al., J Pharmacol Exp Ther 1995, 272: 681-688; Menargues et al., Ann NY Acad Sci 1995, 763: 494-496; Pigini et al., Bioorg Med Chem 1997, 5: 833-841; Boronat et al., Br J Pharmacol 1998, 125: 175-185; Garcia-Sevilla et al., Ann NY Acad Sci 1999, 881: 392-409).

The first selective imidazoline I₂ agonist to be described is 2-BFI (2-(2-benzofuranyl)-2-imidazoline) (for example Lione et al., Br J Pharmacol 1995, 114: 412P; Lione et al., Eur J Pharmacol 1996, 304: 221-229; Coates et al., Bioorg Med Chem Lett 2000, 10: 605-607).

Microdialysis studies have shown that the administration of SRIs raises the extracellular level of the neurotransmitter serotonin (5-HT) in the brain; the increases observed in the literature in the case of the most potent SRIs reach one and a half times to a maximum of double the normal serotonin level in selected brain areas, even at extremely high doses and chronic administration (for example Fuller, Life Sci 1994, 55: 163-167; Gartside et al., Br J Pharmacol 1995, 115: 1064-1070; Arborelius et al., Naunyn Schmiedebergs Arch Pharmacol 1996, 353: 630-640; Malagie et al., Naunyn Schmiedebergs Arch Pharmacol 1996, 354: 785-790; Hjorth et al., Neuropharmacology 1997, 36: 461-465; Dawson et al., Br J Pharmacol 2000, 130: 797-804; Page et al., J Pharmacol Exp Ther 2002, 302: 1220-1227). This increase in serotonergic neurotransmission is regarded as the cause of the therapeutic efficacy of the serotonin reuptake inhibitors (for example Heninger et al., Pharmacopsychiatry 1996, 29: 2-11).

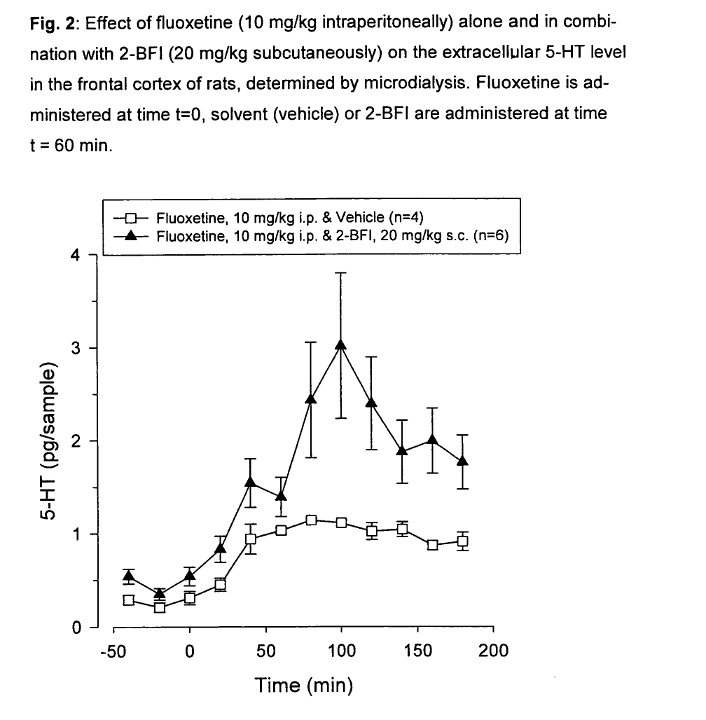

During the work on the present invention, the small increase known from the literature on use of SRIs alone was confirmed. Thus, for example, only a slight increase in the extracellular 5-HT level above the base line is observed in the case of fluoxetine (10 mg/kg intraperitoneally) (FIG. 2).

The object was therefore to increase this effect of SRIs on the extracellular 5-HT level.

Surprisingly, it has now been found by the inventors of the present patent application that an increase of this type can be achieved in a significant manner by administering the SRIs in combination with imidazoline I₂ agonists. This clearly synergistic interaction of the two classes of active ingredient opens up new therapeutic possibilities.

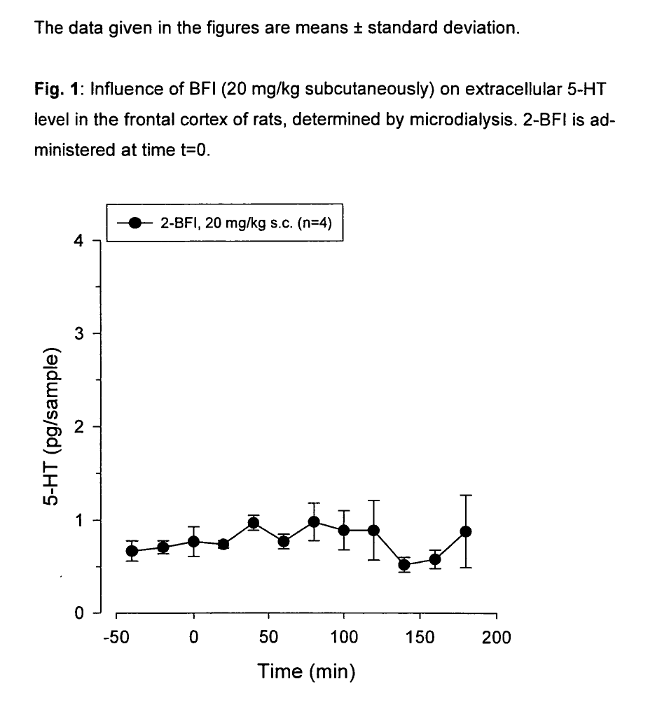

This finding is all the more amazing since imidazoline I₂ agonists alone have no effect on the extracellular 5-HT level in the frontal cortex of rats, as has been shown through the example of 2-(2-benzofuranyl)-2-imidazoline (2-BFI) (20 mg/kg subcutaneously) (FIG. 1).

By contrast, the extracellular 5-HT level is raised approximately three-fold if identical doses of fluoxetine and 2-BFI are combined (FIG. 2).

Such an increase cannot be achieved with fluoxetine alone—even at a relatively high dose (see, for example, Artigas et al., Trends Neurosci 1996; 19: 378-383).

The interaction of the prototypical serotonin reuptake inhibitor (SRI) fluoxetine with the selective imidazoline I₂ receptor ligand 2-(2-benzofuranyl)-2-imidazoline (2-BFI) has been investigated—as described below—by means of microdialysis studies and with reference to animal models of depression and anxiety states.

### 1. Microdialysis Studies

As can be seen from FIG. 1, 2-BFI alone (20 mg/kg subcutaneously) has no effect on the extracellular 5-HT level in the frontal cortex of rats, whereas fluoxetine alone (10 mg/kg intraperitoneally) slightly raises the extracellular 5-HT level (FIG. 2). By contrast, the extracellular 5-HT level is raised approximately three-fold if identical doses of fluoxetine and 2-BFI are combined (FIG. 2).

### 2. Animal Model of Anxiety States

A typical animal model of anxiety states is the “marble burying test” (for example Njung'e and Handley, Br J Pharmacol 1991, 104: 105-112), which has, in particular, specificity for obsessive-compulsive disorders and obsessive-compulsive spectrum disorders. The experimental device consists of a cube-shaped box open at the top in which 25 clear glass marbles are arranged at uniform separations on a 5 cm deep layer of sawdust. Mice are placed individually in the test box for 30 minutes. The number of marbles buried in the sawdust during the test duration of 30 minutes serves as a measure of anxiety. Untreated mice bury the most marbles in the sawdust, anxiolytics reduce the number of buried marbles.

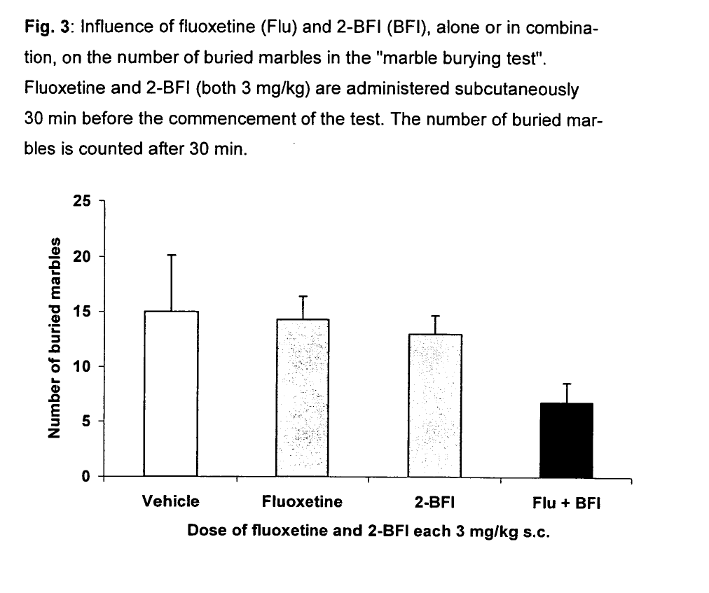

In the test of the individual substances, 2-BFI and fluoxetine (both 3 mg/kg subcutaneously) reduced the number of buried marbles only slightly compared with the vehicle-treated mice, whereas the combination of the two active ingredients at the same dose virtually doubles this number (FIG. 3).

### 3. Animal Model of Depression

A typical animal model of depression is the “forced swimming test” (R. D. Porsolt et al., Nature 1977; 266(5604): 730-732), which is carried out with rats.

This model is based on the behaviour known as “behavioural despair”, which the animals show in a hopeless situation known to them: If the rats are placed in a water-filled vessel (usually for 15 minutes) from which they cannot escape, they cease their attempts to escape from the situation after a certain time and remain in immobility or make only the most necessary swimming movements. If the rats are subjected to this situation again the next day, they recognise the hopelessness of the situation and remain in immobility for the majority of the time, and the time which the animals spend in immobility (which is regarded as surrogate for depression) during the 5-minute test duration serves as a measure of the depression. Anti-depressants shorten this immobility time.

Fluoxetine (5 mg/kg perorally) and 2-BFI (3 and 10 mg/kg perorally) are administered—individually or combined—either in accordance with a repeated acute scheme (fluoxetine 24 h, 5 h and 2 h; 2-BFI 24 h, 5 h and 1 h before commencement of the test) or in accordance with a subchronic scheme (fluoxetine or 2-BFI once daily for 7 days, on the final day fluoxetine 2 h and 2-BFI 1 h before commencement of the test).

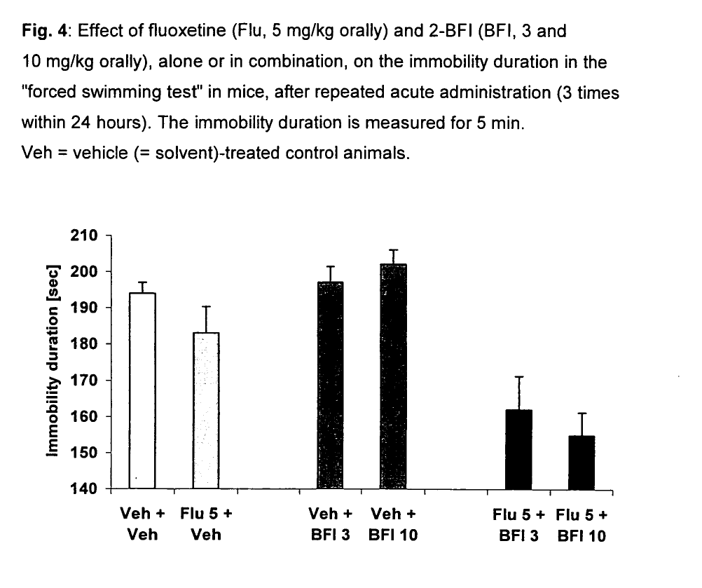

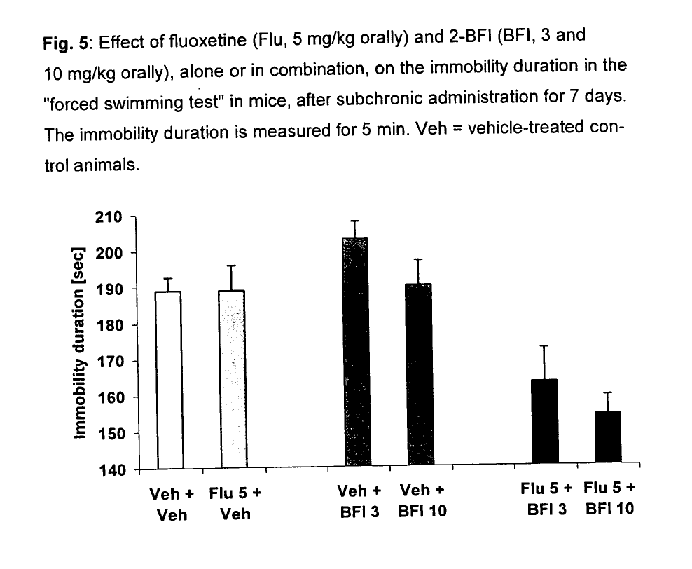

In the repeated acute administration scheme, 2-BFI alone does not reduce the immobility phase, fluoxetine alone shows only a negligible effect. By contrast, the combinations of 2-BFI and fluoxetine give rise to a considerable reduction in the immobility phase, which is dose-dependent for 2-BFI (FIG. 4). Virtually identical effects are also observed in the subchronic scheme (FIG. 5).

The present invention thus relates to a substance and/or a substance mixture which have serotonin reuptake-inhibiting and imidazoline I₂ receptor-agonistic properties and/or solvates, stereoisomers and pharmaceutically usable derivatives thereof, including mixtures thereof in all ratios.

In particular, the present invention relates to a substance which inhibits serotonin reuptake and at the same time binds agonistically to the imidazoline I₂ receptor.

The present invention furthermore relates to a substance which inhibits serotonin reuptake and at the same time binds agonistically to the imidazoline I₂ receptor, and/or substance mixture comprising one or more SRIs and one or more imidazoline I₂ agonists, in particular in its property as medicament, and to corresponding pharmaceutical compositions comprising this substance mixture and optionally further medicament active ingredients (preferably CNS-active ingredients).

In a preferred embodiment, the substance mixture or the pharmaceutical composition comprises fluoxetine as SRI and 2-BFI as imidazoline I₂ agonist.

The invention furthermore relates to the use of a said substance and/or substance mixture for the preparation of a medicament for the treatment or prophylaxis of diseases in which the inhibition of serotonin reuptake and the agonistic binding of active ingredients to the imidazoline I₂ receptor results in improvement of the clinical picture. The diseases are, in particular, depression, obsessive-compulsive disorders (OCDs), obsessive-compulsive spectrum disorders (OCSDs) and anxiety states.

The present invention likewise relates to a pharmaceutical composition according to the invention which, besides the substance and/or substance mixture according to the invention, optionally excipients and/or adjuvants and—optionally—further active ingredients, preferably CNS-active ingredients.

For their preparation, the medicaments can be brought into a suitable dosage form together with at least one solid, liquid and/or semi-liquid excipient or adjuvant and optionally in combination with one or more further active ingredient(s).

In the treatment according to the invention, the substance and/or the substance mixture is generally administered analogously to known preparations, preferably in doses between about 0.1 and 500 mg, in particular between 5 and 300 mg, per dosage unit. The daily dose is preferably between about 0.01 and 250 mg/kg, in particular between 0.02 and 100 mg/kg, of body weight.

The substance and/or substance mixture here is preferably administered in doses between about 1 and 500 mg, in particular between 5 and 100 mg, per dosage unit. The daily dose is preferably between about 0.02 and 10 mg/kg of body weight. However, the specific dose for each particular patient depends on a very wide variety of factors, for example on the efficacy of the specific compound employed, on the age, body weight, general state of health, sex, on the diet, on the time and method of administration, on the excretion rate, medicament combination and severity of the particular disease to which the therapy applies. Oral administration is preferred.

In the case of a substance mixture, the two components of the substance mixture can can be dosed in relation to one another, but also independently.

The pharmaceutical compositions according to the invention can be employed as medicaments in human and veterinary medicine. Suitable carrier substances are organic or inorganic substances which are suitable for enteral (for example oral), parenteral or topical administration and do not react with the novel compounds, for example water, vegetable oils, benzyl alcohols, polyethylene glycols, gelatine, carbohydrates, such as lactose or starch, magnesium stearate, talc, Vaseline. Suitable for enteral administration are, in particular, tablets, dragees, capsules, syrups, juices, drops or suppositories, suitable for parenteral administration are solutions, preferably oily or aqueous solutions, furthermore suspensions, emulsions or implants, suitable for topical application are ointments, creams or powders. The novel compounds may also be lyophilised and the resultant lyophilisates used, for example, for the preparation of injection preparations.

The compositions indicated may be sterilised and/or comprise adjuvants, such as lubricants, preservatives, stabilisers and/or wetting agents, emulsifiers, salts for modifying the osmotic pressure, buffer substances, colorants, flavours and/or aroma substances. If desired, they may also comprise one or more further active ingredients, for example one or more vitamins.

The following examples relate to pharmaceutical compositions:

### EXAMPLE A1

### Injection Vials

A solution of 100 g of a substance and/or substance mixture according to the invention and 5 g of disodium hydrogenphosphate in 3 l of bidistilled water is adjusted to pH 6.5 using 2 N hydrochloric acid, sterile filtered, transferred into injection vials, lyophilised and sealed under sterile conditions. Each injection vial contains 5 mg of substance mixture.

### EXAMPLE A2

### Suppositories

A mixture of 20 g of a substance and/or substance mixture according to the invention is melted with 100 g of soya lecithin and 1400 g of cocoa butter, poured into moulds and allowed to cool. Each suppository contains 20 mg of substance mixture.

### EXAMPLE A3

### Solution

A solution is prepared from 1 g of a substance and/or substance mixture according to the invention, 9.38 g of NaH₂PO₄×2 H₂O, 28.48 g of NaH₂PO₄×12 H₂O and 0.1 g of benzalkonium chloride in 940 ml of bidistilled water. The pH is adjusted to 6.8, and the solution is made up to 1 l and sterilised by irradiation. This solution can be used in the form of eye drops.

### EXAMPLE A4

### Ointment

500 mg of a substance and/or substance mixture according to the invention are mixed with 99.5 g of Vaseline under aseptic conditions.

### EXAMPLE A5

### Tablets

A mixture of 1 kg of a substance and/or substance mixture according to the invention, 4 kg of lactose, 1.2 kg of potato starch, 0.2 kg of talc and 0.1 kg of magnesium stearate is pressed to give tablets in a conventional manner in such a way that each tablet contains 10 mg of substance mixture.

### EXAMPLE A6

### Dragees

Tablets are pressed analogously to Example E and subsequently coated in a conventional manner with a coating of sucrose, potato starch, talc, tragacanth and dye.

### EXAMPLE A7

### Capsules

2 kg of a substance and/or substance mixture according to the invention are introduced into hard gelatine capsules in a conventional manner in such a way that each capsule contains 20 mg of the substance mixture.

### EXAMPLE A8

### Ampoules

A solution of 1 kg of a substance and/or substance mixture according to the invention in 60 l of bidistilled water is transferred into ampoules, lyophilised under sterile conditions and sealed under aseptic conditions. Each ampoule contains 10 mg of substance mixture.

1. Substance which inhibits serotonin reuptake and at the same time binds agonistically to the imidazoline I₂ receptor and/or solvates, stereoisomers and pharmaceutically usable derivatives thereof, including mixtures thereof in all ratios.

2. Substance mixture comprising one or more compounds from the group of the serotonin reuptake inhibitors (SRIs) and/or solvates, stereoisomers and pharmaceutically usable derivatives thereof, including mixtures thereof in all ratios, and one or more compounds from the group of the imidazoline I₂ agonists and/or solvates, stereoisomers and pharmaceutically usable derivatives thereof, including mixtures thereof in all ratios.

3. Substance mixture according to claim 2, comprising fluoxetine and/or solvates, stereoisomers and pharmaceutically usable derivatives thereof, including mixtures thereof in all ratios, and one or more compounds from the group of the imidazoline I₂ agonists and/or solvates, stereoisomers and pharmaceutically usable derivatives thereof, including mixtures thereof in all ratios.

4. Substance mixture according to claim 2, comprising one or more compounds from the group of the serotonin reuptake inhibitors (SRIs) and/or solvates, stereoisomers and pharmaceutically usable derivatives thereof, including mixtures thereof in all ratios, and 2-(2-benzofuranyl)-2-imidazoline (2-BFI) and/or solvates, stereoisomers and pharmaceutically usable derivatives thereof, including mixtures thereof in all ratios.

5. Substance mixture according to claim 2, comprising fluoxetine and/or solvates, stereoisomers and pharmaceutically usable derivatives thereof, including mixtures thereof in all ratios, and 2-BFI and/or solvates, stereoisomers and pharmaceutically usable derivatives thereof, including mixtures thereof in all ratios.

6. Substance or substance mixture according to claim 1 as medicament.

7. Pharmaceutical composition, characterised by a content of a substance or substance mixture according to claim 1 and optionally excipients and/or adjuvants.

8. Use of a substance or substance mixture according to claim 1 for the preparation of a medicament for the treatment or prophylaxis of diseases in which the inhibition of serotonin reuptake and the agonistic binding to the imidazoline I₂ receptor results in improvement of the clinical picture.

9. Use according to claim 8, characterised in that the diseases are depression, obsessive-compulsive disorders (OCDs), obsessive-compulsive spectrum disorders (OCSDs) and other anxiety states.
